(19) United States
(12) Patent Application Publication
(10) Pub. No.: US 2008/0195228 A1
(43) Pub. Date: Aug. 14, 2008

(54) Bowel Implant

(75) Inventors: Jorgen Uno, Linhamn (SE); Lars Magnus Bjursten, Limhamn (SE); Zhongquan Qi, Malmo (SE)

Correspondence Address: WRB-IP LLP, 1217 KING STREET, ALEXANDRIA, VA, 22314, US

(73) Assignee: Sinova Safetech Innovation AB, Malmo (SE)

(21) Appl. No.: 11/917,000

(22) PCT Filed: Mar. 28, 2006
(86) PCT No.: PCT/SE06/00378

§ 371 Date: Dec. 9, 2007

(30) Foreign Application Priority Data
Jun. 16, 2005 (SE) 0501380-0

(51) Int. Cl. A61F 2/04

(52) U.S. Cl. 623/23.71

(57) ABSTRACT

An apparatus for operation into the intestine of humans and animals includes a flexible, double-walled tube. The outer wall of the tube is porous for the inward growth of blood vessels, while its inner wall is tight for a barrier effect against the contents of the intestine. A degradable, support structure is advantageously disposed inside the inner wall of the tube.

### BACKGROUND AND SUMMARY

The present invention relates to an apparatus for operation into human and animal intestines, and comprising a flexible, double-walled tube.

People suffering from intestinal diseases, tumours or different types of injury are today obliged to undergo surgical removal of parts of the intestines. In this instance, a colostomy is applied to make possible emptying of the intestine. This often entails that the residual intestinal end discharges through the abdominal wall and the intestinal contents are collected in a colostomy bag disposed exteriorly. The colostomy container may be designed in different ways, and even disposed inside the abdominal cavity, but is generally experienced as inconvenient and troublesome. It is thus desirable in the art to maintain as far as is possible a natural intestine. In many cases, this has been possible when only short pieces of the intestine have necessitated removal, but when longer pieces have been removed, it has hitherto not been possible to resurrect a functional intestine with a satisfactory nutrient uptake and functioning peristalsis.

U.S. Pat. No. 4,747,849 discloses one example of an operable, double-walled tube which is intended to replace an injured oesophagus. The double-walled tube also includes a helical band in order to keep the tube open and transfer deformations from the outer wall to the inner wall. Since the tube is intended for the oesophagus, it is quite rigid and, for this reason, would probably not function particularly well as a substitute for a length of the intestine, since the intestine movements—the peristalsis—constitute a vitally important part of the intestinal function. Moreover, the environment within the intestine is totally different from that prevailing in the oesophagus. Among other things, the intestinal content remains in the intestine for considerably longer periods than those periods of time during which food passes through the oesophagus. Finally, the prime function of the intestine is to absorb nutrient substances from the contents of the intestine, which takes place with the aid of cells on the inside of the intestine, in the so-called intestinal villi, which in turn are connected to blood vessels for further transport of the absorbed nutrient substances. The arrangement described in the above cited United States Patent completely lacks such functions.

It has hitherto been possible to cultivate colonies of intestinal cells in a laboratory environment. In this instance, use is made of a so-called tissue graft on which the intestinal cells have been able to grow and propagate. However, it should be kept in mind that the laboratory environment is typically considerably less aggressive than the environment in the intestine of a living human or a living animal. Since the cultivation often takes place in a nutrient solution, the supply of nutrient to the cells is no major problem either. On the other hand, it has not hitherto been possible to realise a functioning supply of nutrient to the cells in a living being. The conclusion is that a viable, functioning intestine cannot be produced in a laboratory environment.

There is thus a need in the art to be able to realise a functional, natural-like permanent intestinal portion in a human patient or alternatively an animal.

An apparatus according to an aspect of the present invention is characterised in that the outer wall of the double-walled tube is porous for allowing the inward growth of blood vessels, while its inner wall is impermeable for a barrier effect against the contents of the intestine.

### BRIEF DESCRIPTION OF THE ACCOMPANYING DRAWINGS

The present invention will now be described in greater detail hereinbelow, with reference to the accompanying Drawings. In the accompanying Drawings:

FIG. 1 is a perspective view of the implant according to the present invention;

FIG. 2 is a view corresponding to that of FIG. 1 where the implant has been operated into the mesentery;

FIG. 3 is a view corresponding to that of FIGS. 1 and 2, where the implant has been operated in between two intestinal ends; and

FIG. 4a-c are sectional views taken along the line A-A—in FIG. 3 in three different time phases.

### DETAILED DESCRIPTION

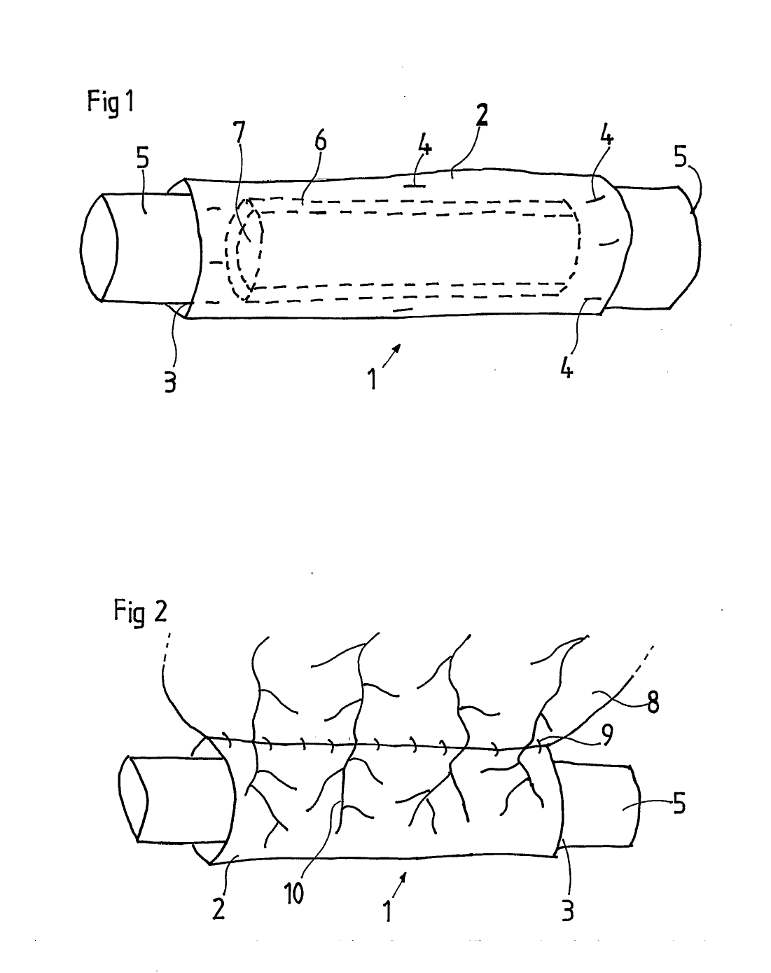

FIG. 1 shows the implant in perspective from the side. The apparatus is tubular and flexible so that it may be bent and compressed in principle in all directions, substantially corresponding to the properties of a natural intestine.

The implant 1 has an outer wall 2 and an inner wall 3. Both walls 2, 3 are tubular and flexible in the above-mentioned manner. The outer wall 2 is disposed on the outside of the inner wall 3 and they are secured to one another, preferably in a releasable manner. In the preferred embodiment, the union between them is put into effect on the one hand by a degradable fibrin glue between the outer wall 2 and the inner wall 3 and, on the other hand, by means of individual sutures 4.

The inner wall 3 is longer than the outer wall 2, so that the implant 1 is provided with collars 5 of the inner wall 3 at its ends. The collars 5 will facilitate the securing of the apparatus 1 in the intestine of a human or an animal.

The outer wall 2 is manufactured from a porous material with many small openings which permit an inward growth of blood vessels, nerve fibres and other cells on and through the material. This material is also designated tissue graft. In one preferred embodiment, the tissue graft is manufactured from the polyester material Dacron™ or the like. The employed material may readily be degradable after a time in the body, but a suitably selected non-degradable material would also function well.

The size of the holes or the pores in the material is important. They must have a transverse dimension of at least 50 micrometers. The distance between the holes should naturally be as small as possible. Consequently, a knitted material is advantageously employed, since the distance between the holes of the material then does not exceed the thickness of the fibres included. The material is both flexible, in the manner described above, and also elastic in several directions. However, it is possible alternatively to utilise a material which is sufficiently openly woven. A further alternative could be to employ a sheet-shaped material with a number of perforations or foraminations which satisfy the requirements on pore size. However, a certain degree of elasticity is essential, since the material must have properties corresponding to a natural intestine.

In one preferred embodiment, the tubular outer wall 2 is manufactured so that it is jointless, i.e. manufactured employing a method which corresponds to the knitting of a sock. In trials, it has however proved possible to realise the tubular wall 2 also from a planar material whose opposing edges are united together to form a seam or joint.

The inner wall 3 consists of a tight or impervious layer of a suitable material. One such material in the preferred embodiment is a silicon rubber sheet which is both tight and flexible as well as being elastic. Since the inner wall 3 will come into contact with the contents of the intestine, in order to protect the area outside the inner wall 3, it is important that the material is resistant to the stomach- and intestinal juices and the enzymes existing therein, at least during the desired service life, in general one or a few weeks. Thereafter, the inner wall 3 will have been released from the outer wall 2 and will be voided “the natural way”.

The outer wall 2 and the inner wall 3 are secured to one another by means of a releasable connection. In the preferred embodiment, the connection consists, on the one hand, of a fibrin glue and, on the other hand, of a few degradable sutures. The fibrin glue is commercially available and is mixed immediately before use. The glue contains, on the one hand, fibrinogen and, on the other hand, the enzyme prothrombin for the formation of fibrin. The fibrin glue has proved to have an effect which promotes the growth of cells, which is a major advantage since as rapid a cell growth as possible is desirable on and in the outer wall 2.

The sutures 4 are not particularly numerous, not least because it is not desirable to perforate the inner wall 3 unnecessarily. The sutures are preferably so-called monofilament sutures, i.e. sutures which consist of a single thread or fibre, which reduces the risk of infection. The material in the sutures may be polypropylene, which is not degradable, but may accompany the inner wall 3 when this is rejected. The suture may also be degradable, for example polylactide, which is degradable in contact with water. The suture will then disappear completely.

Inside the implant 1, there is disposed a support structure 6 which is manufactured of a material which breaks down in time, such as, for example, starch. The support structure 6 displays a substantially annular cross section, and has an extent in the longitudinal direction which is of the same order of magnitude as the implant, possibly slightly shorter. This entails that the support structure, which is intimated by broken lines in FIG. 1, in the preferred embodiment is in the form of an elongate tube. It is important that the support structure 6 be permeable with a central channel 7 so that the contents of the intestine may pass therethrough. The function of the support structure is to keep the implant open so that normal tissue may grow on both the outside and the inside of the implant 1. If this were not the case, there is a risk that the intestine newly formed on the implant will not assume a natural configuration and that its permeability will be highly restricted, since its central opening will be far too tight. In order to attain the purpose of the support structure 6, it is, however, conceivable that several shorter support structures be disposed at a certain spacing from one another.

The implant 1 may be wholly or partly impregnated with titanium oxide in order to render the structure more stable and in order to increase the body's acceptance of the alien material.

The implant 1 is employed as follows: in a first stage, the implant 1 is fixedly operated in the mesentery (omentum) in order to realise a vessel supply and an outer layer of cells on the implant. The mesentery comprises folds of the peritoneum and is rich in blood vessels and nerves. In addition, the mesentery fixes the intestine to the rear wall of the abdominal cavity.

FIG. 2 shows a part of the mesentery 8 in which the implant 1 is secured by means of sutures 9. Cells from the mesentery 8 have begun to grow into the outer wall 2 and are well supplied with nutrient from the blood vessels 10 which have similarly grown into the outer wall 2. The result will be that a tube of living epithelial cells has been built up starting from the outer wall 2.

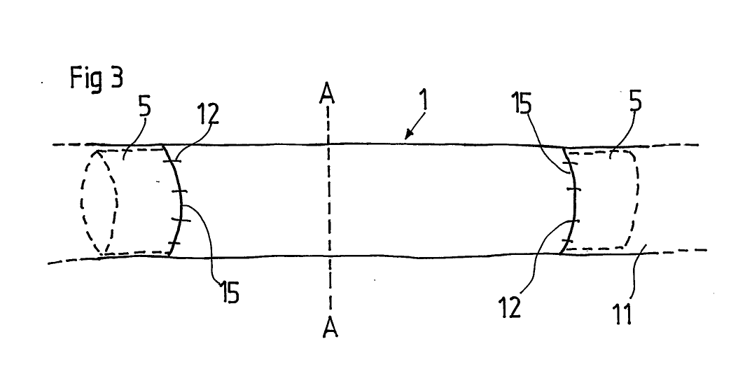

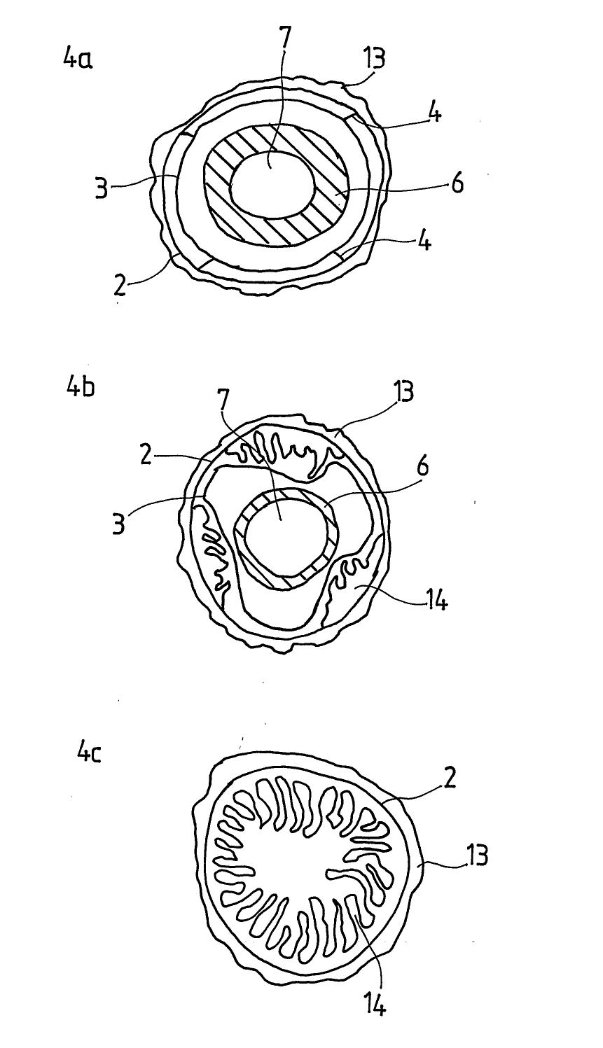

FIG. 3 shows the second stage in the employment of the implant 1. The implant 1 covered by epithelial cells is operated in between two healthy intestinal ends 11. While this is not shown in FIG. 2, both the intestinal ends 11 and the implant 1 covered by cells has a connection to the mesentery 8 for continued supply of blood and nutrient. The collars 5 are utilised for fixing the healthy ends of the intestine 11. This fixing is put into effect with the aid of a layer of fibrin glue which is coated on the outside of the collars 5 and secured against the inner wall of the healthy intestinal ends 11. The joint 15 between the healthy intestinal ends and those cells which cover the outer wall 2 of the implant 1 are moreover stitched together using sutures 12. As a result, the inside of the healthy intestinal end 11 will lie under the collar 5 which entails, since the collar 5 is tightly glued against the inside, that the joint 15 will be protected during the healing process. At the same time, cells from the inside of the healthy intestinal ends 11 will begin to grow inside the inner wall 3 and will in due course meet, in which event the intestine has thus been lengthened a distance. This growth takes place constantly under the protection of the inner wall 3 of the implant 1.

FIGS. 4a-c show this process in the cross section taken along the line A-A in FIG. 3. Those cells which grow in from the insides of the healthy intestinal ends 11 withstand contact with the contents of the intestine, since they are naturally adapted to be located on the inside of the intestine. Those cells which are located on the outside of the implant, i.e. on the outside of the outer wall 2 are, however, highly sensitive to the contents of the intestine. Thus, the inner wall 3 protects both the sensitive outer cells and the less sensitive inner cells before they have subsequently become established.

FIG. 4a shows the inner tube 2 covered by outer epithelial cells against which outer tube the tight inner tube 3 is secured by means of fibrin glue and/or sutures 4. In order to keep both the outer wall 2 and the inner wall 3 in an open, tensioned position for an optimum growth of both outer and inner cells, the support structure 6 is disposed centrally in the implant 1.

Even if the support structure 6 allows passage of the contents of the intestine through its central channel 7, it is nevertheless not intended to remain permanently in the implant 1. FIG. 4b shows how the support structure 6 has begun to break down, both from its outside facing towards the inner wall 3 and from the inside of the central channel 7. At the same time, a growth of inner cells 14 can be seen between the outer wall 2 and the tight inner wall 3. Since the fibrin glue and possibly also the sutures are slowly degradable, these will not impede this growth. The consequence will be that when the inside of the outer wall 2 is covered by inner cells 14, the inner wall 3 has lost contact with the outer wall 2. The inner cells 14 are then so well established that they withstand the contact with the contents of the intestine. Moreover, the layer of inner cells 14 will protect the outer cells 13 from the harmful effects of the contents of the intestine. The support structure 6 is needed less and less and, in due course, will be completed dissolved by the contents of the intestine. The residue of the support structure 6 will then be removed together with the contents of the intestine and the inner wall 3.

FIG. 4c shows the section through the resulting intestinal extension. In this situation, the only matter which is remaining in position of the implant 1 is the outer wall 2 through which both outer cells 13 and inner cells 14 have grown as well as blood vessels in order to supply them. Possibly, the outer wall 2 will be broken down and disappear, on condition that it is manufactured from a degradable material.

The implant 1 may also be secured in the abdominal muscle instead of in the mesentery 8. In such an event, cells from the abdominal muscular structure will grow into the outer wall 2 and, in this case, the intention is principally to realise a colostomy container which is worn internally. In such an application, a stability may be desirable to a higher degree, since such a container need not be as easily movable as a part of an intestine. For example, such stability may be achieved with the aid of impregnation or other reinforcement of the implant 1 with titanium oxide. Naturally, the size and configuration of the implant 1 may need to be adapted to its practical application. The inside of the final internal colostomy container will come into contact with the contents of the intestine and, as a result, the same durable inside as that existing in the intestine is desirable. Inner cells 14 may be caused to grow into position in the manner corresponding to that described above, but this growth will, in such an event, only take place from one direction, i.e. from that end where the intestine discharges in the contemplated colostomy container. At that end of the colostomy container which is connected to an opening to the outside, there may be provided some form of lid or valve in the abdominal wall.

The invention may be varied further without departing from the scope of the appended Claims.

1. An apparatus for operation into the intestine of humans and animals, comprising a flexible, double-walled tube, wherein the outer wall of the tube is porous for the inward growth of blood vessels, while its inner wall is tight for a barrier function against contents of the intestine.

2. The apparatus as claimed in claim 1, wherein a degradable, support structure is disposed inside the inner wall of the tube.

3. The apparatus as claimed in claim 1, wherein the inner and outer walls of the tube are releasably connected to one another.

4. The apparatus as claimed in claim 3, wherein the inner and outer walls are connected to one another by means of fibrin-containing glue.

5. The apparatus as claimed in claim 3, wherein the inner and outer walls are connected to one another by means of degradable sutures.

6. The apparatus as claimed in claim 1, wherein the inner wall is longer than the outer so that the inner wall forms collars at the end of the tube.

7. The apparatus as claimed in claim 1, wherein the outer wall is manufactured from a degradable material.

8. The apparatus as claimed in claim 1, wherein the outer wall is a knitted fabric material.

9. The apparatus as claimed in claim 1, wherein the outer wall is a woven material.

10. The apparatus as claimed in claim 1. wherein the outer wall is a perforated material.

11. The apparatus as claimed in claim 1, wherein the tube is impregnated with titanium oxide.

12. The apparatus as claimed in claim 2, wherein the inner and outer walls of the tube are releasably connected to one another.

13. The apparatus as claimed in claim 12, wherein the inner and outer walls are connected to one another by means of fibrin-containing glue.

14. The apparatus as claimed in claim 12, wherein the inner and outer walls are connected to one another by means of degradable sutures.

15. The apparatus as claimed in claim 2, wherein the inner wall is longer than the outer so that the inner wall forms collars at the end of the tube.

16. The apparatus as claimed in claim 2, wherein the outer wall is manufactured from a degradable material.

17. The apparatus as claimed in claim 2, wherein the outer wall is a knitted fabric material.

18. The apparatus as claimed in claim 2, wherein the outer wall is a woven material.

19. The apparatus as claimed in claim 2, wherein the outer wall is a perforated material.

20. The apparatus as claimed in claim 2, wherein the tube is impregnated with titanium oxide.
